(19) United States
(12) Patent Application Publication
(10) Pub. No.: US 2026/0174385 A1
(43) Pub. Date: Jun. 25, 2026

(54) System and Method for Medication Titration Using Phone Health Data (Walking/Balance Metrics)

(71) Applicant: Stanley G. Van Meter, II, Lake Mary, FL (US)

(72) Inventor: Stanley G. Van Meter, II, Lake Mary, FL (US)

(21) Appl. No.: 19/426,830

(22) Filed: Dec. 19, 2025

Related U.S. Application Data
(60) Provisional application No. 63/736,710, filed on Dec. 20, 2024.

(51) Int. Cl. A61B 5/00; A61B 5/11; G16H 10/60; G16H 20/17

(52) U.S. Cl. CPC A61B 5/4839; A61B 5/112; A61B 5/4023; A61B 5/6898; A61B 5/7267; A61B 5/746; A61B 5/747; G16H 10/60; G16H 20/17

(57) ABSTRACT

A system and method for managing medication titration using real-time walking and balance data collected from mobile electronic devices are disclosed. Smartphone sensors, including accelerometers and gyroscopes, capture mobility metrics such as gait speed, stride characteristics, postural stability, and fall risk. The mobility data is integrated with additional physiological metrics and a patient's electronic medical records. Analytical algorithms, including machine-learning models, evaluate changes in mobility to identify correlations between medication usage and functional performance. Based on detected changes, the system generates alerts and recommendations to assist healthcare providers in adjusting medication dosages. The invention supports proactive and personalized medication management, particularly for medications that affect balance or fall risk, and improves patient safety by enabling data-driven clinical decision-making using real-world mobility insights.

### FEDERALLY SPONSORED RESEARCH

Not Applicable

### SEQUENCE LISTING OR PROGRAM

Not Applicable

### TECHNICAL FIELD OF THE INVENTION

The present invention relates to healthcare technology, specifically to systems and methods for using real-time walking and balance data collected from mobile devices to manage medication titration. More specifically, the present invention integrates mobility metrics with electronic medical records and advanced algorithms to provide personalized and data-driven adjustments to medication dosages.

### BACKGROUND OF THE INVENTION

Effective management of medication titration is critical for patients with conditions influenced by mobility and balance, such as Parkinson's disease, hypertension, and diabetes. Traditionally, medication dosages are adjusted based on periodic assessments in clinical settings or static data from wearable or monitoring devices. However, these approaches often fail to capture the dynamic, real-world variations in a patient's mobility and balance, which are essential indicators of their overall health and response to medication.

Recent advancements in mobile health technology have enabled the collection of walking and balance metrics through smartphone sensors, such as accelerometers and gyroscopes. Despite these capabilities, current systems primarily focus on fitness tracking or generalized health monitoring. They do not integrate this valuable data with medication management processes, leaving a significant gap in utilizing real-time mobility insights for precise and proactive dosage adjustments.

The lack of systems that leverage real-time walking and balance data for medication titration often leads to suboptimal treatment plans. For instance, medications known as Fall Risk Increasing Drugs (FRIDs) can inadvertently increase the likelihood of falls if dosages are not closely monitored and adjusted based on the patient's mobility metrics. Similarly, conditions like Parkinson's disease could benefit from dosage increases when improvements in gait stability are observed, but such insights are rarely acted upon due to the absence of integrated systems.

This invention addresses these challenges by providing a comprehensive system that collects real-time mobility data via smartphones, integrates it with patient health records, and uses advanced algorithms to assist healthcare providers in medication titration. The system offers the potential to improve patient outcomes by delivering personalized, data-driven insights for medication adjustments while reducing risks associated with static or outdated evaluation methods.

### SUMMARY OF THE INVENTION

The present invention provides a system and method for medication titration management using real-time walking and balance metrics collected through mobile devices. By leveraging smartphone sensors such as accelerometers and gyroscopes, the system monitors a patient's mobility data, including gait speed, stride length, postural stability, and fall risk. This data is analyzed alongside other health metrics, such as heart rate and blood pressure, and integrated with the patient's electronic medical records (EMRs). The system utilizes advanced algorithms, including machine learning models, to identify significant changes in mobility that may indicate the need for adjustments in medication dosages.

The invention enables proactive healthcare by providing alerts and recommendations to healthcare providers when walking or balance metrics suggest potential adverse medication effects or the need for dosage adjustments. For example, the system may flag a decline in balance stability as a signal to reduce medications like sedatives or antihypertensives. Conversely, improvements in mobility may prompt recommendations to increase medication dosages for conditions such as Parkinson's disease. Alerts are generated in real-time and include actionable insights for clinicians, enhancing the precision of medication management.

The invention is particularly beneficial for managing medications that impact balance, such as Fall Risk Increasing Drugs (FRIDs). By integrating real-world mobility data with clinical information, the system offers personalized, dynamic medication titration, reducing fall risk and improving patient outcomes. Additionally, aggregated data can be used to identify broader trends, providing valuable insights for public health initiatives and clinical research.

This system represents a significant advancement in healthcare technology by bridging the gap between real-time mobility data and clinical decision-making. It improves medication management, enhances patient safety, and supports healthcare providers with actionable, data-driven insights.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized, and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

The present invention relates to a novel system and method that integrates phone-based walking and balance metrics to assist in medication titration for conditions influenced by mobility and balance, such as Parkinson's disease, hypertension, and diabetes. By leveraging real-time data collected from smartphone sensors, the system provides a dynamic and precise approach to adjusting medication dosages based on a patient's physical condition. Traditional medication titration often relies on static clinical data, which fails to capture the continuous fluctuations in a patient's mobility. This invention fills that gap, offering a comprehensive solution for healthcare providers to manage medications effectively and in real-time.

The system collects walking and balance data through smartphone applications, such as the Balance EQ app, which utilizes embedded sensors like accelerometers and gyroscopes. Key data points include gait speed, stride length, step count, postural stability, and fall risk assessments. The data is collected continuously or at intervals, depending on patient-specific settings, and transmitted securely to the system's processing unit. Additionally, the system integrates this mobility data with other health metrics, such as heart rate, blood pressure, and glucose levels, through APIs like Apple HealthKit or Google Fit. This integrated data is stored and analyzed alongside the patient's electronic medical records (EMRs), focusing on medications with known impacts on balance, such as Fall Risk Increasing Drugs (FRIDs).

To determine when medication adjustments are necessary, the system applies machine learning models or preset algorithms to identify significant changes in walking or balance metrics. For example, if a decline in postural stability is detected, the system flags this data for review and may recommend reducing medications such as sedatives or antihypertensives. Conversely, improvements in gait speed or stability may prompt the system to suggest increasing medications for conditions like Parkinson's disease. These recommendations are supported by evidence from the patient's historical data, ensuring that the titration process is both personalized and data-driven.

An integrated alert system ensures that healthcare providers are notified of significant changes in the patient's walking and balance data. Alerts include specific recommendations for medication adjustments or requests for additional data, such as blood pressure or heart rate, to confirm whether a dosage change is necessary. For instance, a patient on antihypertensive medication exhibiting balance deterioration may receive a flagged alert, prompting the provider to review the dosage and adjust it to minimize fall risk. Similarly, a Parkinson's patient showing improvement in walking metrics after starting a new medication may receive a recommendation to gradually increase the dosage for enhanced therapeutic outcomes.

The system also provides actionable insights for healthcare providers through data visualization tools that highlight trends and correlations between mobility metrics and medication adjustments. Aggregated and anonymized data from multiple patients can also be analyzed to identify broader population-level trends, providing valuable insights for public health initiatives or clinical research.

This invention has several use case scenarios. For instance, a patient on blood pressure medication who experiences worsening balance could be flagged for a dose reduction to prevent potential falls. Alternatively, a Parkinson's patient whose walking metrics improve after medication initiation could be monitored for continued improvement, with incremental dose increases recommended to maximize efficacy. These real-world applications demonstrate the system's potential to significantly improve patient outcomes by proactively addressing mobility-related side effects of medications.

The system complies with all relevant data privacy regulations, including GDPR and HIPAA. Walking and balance data are anonymized before storage and analysis, ensuring the protection of patient confidentiality. By integrating advanced algorithms, real-time mobility data, and EMRs, this system represents a transformative approach to medication management, offering precision and safety that traditional methods lack. The invention provides a vital tool for optimizing medication dosages, reducing falls, and improving overall patient well-being, making it a valuable innovation in healthcare technology.

The system is set to run on a computing device or mobile electronic device. A computing device or mobile electronic device on which the present invention can run would be comprised of a CPU, Hard Disk Drive, Keyboard, Monitor, CPU Main Memory and a portion of main memory where the system resides and executes. Any general-purpose computer, smartphone, or other mobile electronic device with an appropriate amount of storage space is suitable for this purpose. Computer and mobile electronic devices like these are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Thus, it is appreciated that the optimum dimensional relationships for the parts of the invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

1. A non-transitory computer-readable medium storing instructions that, when executed by a processor, enables a system for managing medication titration using walking and balance data collected from a mobile device, comprising:
a mobile application configured to collect walking and balance metrics from smartphone sensors, including accelerometer and gyroscope data;
a data integration module configured to combine the walking and balance metrics with additional health data, including heart rate, blood pressure, or glucose levels, retrieved via application programming interfaces (APIs) or other external systems;
a machine learning engine or algorithm module configured to analyze the walking and balance metrics to identify patterns or changes indicative of medication efficacy or adverse effects;
an alert generation module configured to create and transmit alerts or recommendations for medication dosage adjustments based on the analysis; and
a communication interface configured to securely transmit the alerts and recommendations to healthcare providers or patients.

2. The system of claim 1, wherein the walking and balance metrics include at least one of gait speed, stride length, step count, postural stability, or fall risk assessments.

3. The system of claim 1, further comprising a data anonymization module to ensure compliance with privacy regulations, including GDPR and HIPAA, by anonymizing collected walking and balance data.

4. The system of claim 1, wherein the additional health data is retrieved via integration with external health monitoring platforms, such as Apple HealthKit or Google Fit.

5. The system of claim 1, further comprising a real-time notification system configured to alert healthcare providers when significant deviations in walking or balance metrics are detected.

6. The system of claim 1, wherein the machine learning engine is trained using historical data sets comprising walking and balance metrics combined with medication dosage records.

7. The system of claim 1, further comprising an adaptive threshold module configured to dynamically adjust sensitivity levels for detecting significant changes in walking and balance metrics based on a patient's historical data and condition.

8. The system of claim 1, wherein the alert generation module is further configured to provide condition-specific medication recommendations for diseases such as Parkinson's, hypertension, or diabetes.

9. The system of claim 1, further comprising a user interface accessible via the mobile application, allowing patients to view personalized insights, medication recommendations, and suggested actions to improve mobility and health outcomes.

10. The system of claim 1, wherein the communication interface is configured to securely transmit medication-related recommendations and alerts to healthcare providers via electronic health record (EHR) systems or other healthcare platforms.

11. A method for managing medication titration using walking and balance data collected from a mobile device, the method comprising:
collecting walking and balance metrics from smartphone sensors, including accelerometer and gyroscope data;
integrating the walking and balance metrics with patient health records and additional health data, including heart rate, blood pressure, or glucose levels;
analyzing the walking and balance metrics using machine learning models or predefined algorithms to detect changes indicative of medication efficacy or adverse effects;
generating alerts and recommendations for medication dosage adjustments based on the analyzed data; and
transmitting the alerts and recommendations to a healthcare provider for review or directly to the patient for action.

12. The method of claim 11, wherein the walking and balance metrics include at least one of gait speed, stride length, step count, postural stability, or fall risk assessments.

13. The method of claim 11, further comprising anonymizing collected walking and balance data for secure storage and compliance with privacy regulations such as GDPR or HIPAA.

14. The method of claim 11, wherein the additional health data is collected via integration with external systems using application programming interfaces (APIs) such as Apple HealthKit or Google Fit.

15. The method of claim 11, further comprising sending real-time notifications to healthcare providers when significant deviations in walking or balance metrics are detected.

16. The method of claim 11, wherein the machine learning models are trained using historical walking and balance data combined with medication dosage patterns.

17. The method of claim 11, further comprising dynamically adjusting sensitivity thresholds for detecting significant changes in walking and balance metrics based on the patient's historical data and condition.

18. The method of claim 11, further comprising generating customized recommendations for medication changes specific to conditions such as Parkinson's disease, hypertension, or diabetes.

19. The method of claim 11, wherein the alerts include suggestions for additional data collection, such as requesting manual entry of symptoms or additional health metrics like blood pressure readings.

20. The method of claim 11, further comprising providing a user interface for patients to view personalized insights, medication recommendations, and potential actions to improve their mobility and health outcomes.
